(19) United States
(12) Patent Application Publication
(10) Pub. No.: US 2006/0002913 A1
(43) Pub. Date: Jan. 5, 2006

(54) Use of histamine and related compounds to treat disorders affecting muscle function

(76) Inventor: Kurt R. Gehlsen, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/158,798

(22) Filed: Jun. 20, 2005

Related U.S. Application Data
(60) Provisional application No. 60/581,872, filed on Jun. 22, 2004.

(51) Int. Cl. A61K 38/44; A61K 31/385; A61K 31/355; A61K 31/375; A61K 31/198; A61K 31/07

(52) U.S. Cl. 424/94.4; 514/458; 514/474; 514/562; 514/725

(57) ABSTRACT

Described herein are compositions and methods for treating and/or preventing disorders affecting skeletal muscular function that are caused by reactive oxygen species in mammals. More specifically, the disclosure relates to the treatment and/or prevention of disorders affecting skeletal muscular function through the administration of histamine and histamine-related compounds.

### CROSS REFERENCE TO RELATED APPLICATIONS

This application claims priority under 35 U.S.C. § 119(e) to U.S. Provisional Application No. 60/581,872, filed on Jun. 22, 2004, entitled USE OF HISTAMINE AND RELATED COMPOUNDS TO TREAT DISORDERS AFFECTING MUSCLE FUNCTION; the disclosure of which is incorporated herein by reference in its entirety.

### BACKGROUND OF THE INVENTION

1. Field of the Invention

Described herein are methods for treating and/or preventing disorders affecting skeletal muscular function caused by oxidative stress and/or inflammation in mammals. More specifically, the disclosure relates to the treatment and/or prevention of disorders affecting skeletal muscular function through the administration of histamine and histamine-related compounds.

2. Description of the Related Art

Oxidative stress, i.e. toxicity inflicted by reactive oxygen species (ROS), is being recognized as a systemic phenomenon in diseases that affect muscle function. The extent of oxidative stress appears to correlate with the severity and stage of disease. The mechanism of action associated with the damage caused by oxidative stress has been implicated in a number of diseases and relates to direct damage of muscle. Examples of such disorders include fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle.

The role of oxidative stress in disorders affecting muscle function is becoming more clear. For example, recent work indicates that reactive oxygen species that leak out of mitochondrial membranes during aerobic respiration have a significant degenerative effect on muscle fibres. Hudson and Franklin, J. Exp. Biol. 205:2297-2303 (2002). In addition, cell-mediated ROS from infiltrating neutrophils or macrophages can release ROS and cause cellular damage. Moreover, these same cells release inflammatory cytokines which affect muscle function. Skeletal muscles are particularly vulnerable to oxidative stress as they use large amounts of oxygen and continuously generate ROS. Marzani et al., Basic Appl. Myol. 14(1):37-44 (2004). Oxidative stress has also been proposed to contribute to the state of immunosuppression at the site of malignant tumors and in chronic viral infections. (See U.S. Pat. Nos. 5,728,378, 6,000,516, and 6,155,266). Lymphocytes residing within or adjacent to tumors display signs of oxidative damage, including a higher degree of apoptosis and a defective transmembraneous signal transduction. The oxidative stress at the site of tumor growth is presumably conveyed by ROS produced by adjacent phagocytic cells (monocyte/macrophages (MO) or neutrophilic granulocytes (GR)). Histamine, an inhibitor of ROS production in phagocytes, is currently used as an adjunct to lymphocyte-activating cytokines (IL-2 and IFN-alpha) with the aim to enhance cytokine efficiency.

The complete reduction of one molecule of O₂ to water is a four-electron process. Oxidative metabolism continually generates partially reduced species of oxygen, which are far more reactive, and hence more toxic than O₂ itself. A one-electron reduction of O₂ yields superoxide ion (O₂⁻); reduction by an additional electron yields hydrogen peroxide (H₂O₂), and reduction by a third electron yields a hydroxyl radical (OH.), and a hydroxide ion. Nitrous oxide (NO), is another interesting reactive oxygen metabolite, produced through an alternative pathway. Hydroxyl radicals in particular are extremely reactive and represent the most active mutagen derived from ionizing radiation. All of these species are generated and must be converted to less reactive species if the organism is to survive.

Particular cells of the immune system have harnessed the toxic effects of ROS as an effector mechanism. Professional phagocytes, polymorphonuclear leukocytes (neutrophils, PMN), monocytes, macrophages, and eosinophils function to protect the host in which they reside from infection by seeking out and destroying invading microbes. These phagocytic cells possess a membrane-bound enzyme system which can be activated to produce toxic oxygen radicals in response to a wide variety of stimuli.

The “increased respiration of phagocytosis” (the respiratory burst) was reported and thought to be a result of increased mitochondrial activity providing additional energy for the processes of phagocytosis. It was later shown that a non-mitochondrial enzymatic system produced the increased levels of oxygen metabolites since the respiratory burst continued even in the presence of mitochondrial inhibitors such as cyanide and antimycin A. In 1968, Paul and Sbarra showed clearly that hydrogen peroxide was produced by stimulated phagocytes and in 1973 Babior and co-workers established that superoxide was a major product of the oxidase. (Paul and Sbarra, Biochim Biophys Acta 156(1):168-78 (1968); Babior, et al., J Clin Invest 52(3):741-4 (1973)). It is now generally accepted that the enzyme is membrane bound, exhibits a preference for NADPH (Kₘ=45 μM) over NADH (Kₘ=450 μM), and converts oxygen to its one electron-reduced product, superoxide.
NADPH+H⁺+2O₂→NADP⁺+2H⁺+2O₂⁻
The hydrogen peroxide arises from subsequent dismutation of the superoxide.
2O₂⁻+2H⁺→H₂O₂+O₂⁻

While there are beneficial effects of these oxygen metabolites, it is clear that inappropriate production of oxygen metabolites can result in severely deleterious effects. For example, inappropriate production of ROS and/or inflammatory cytokines by skeletal muscles can lead to muscle atrophy and free radical damage associated with many degenerative conditions. Several disease states illustrate this point, including various disorders that affect skeletal muscular function, such as, for example, fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle. An effective method to reduce and/or minimize the production and release of ROS and/or inflammatory cytokines in patients suffering from such disorders would be a great boon to medicine and serve to reduce and eliminate a substantial amount of human suffering.

Given the ravaging effects of disorders that affect skeletal muscular function and the only partially successful treatment methods available today, there is a constant demand for improved methods of treating such disorders and reducing damage to the muscular system.

### SUMMARY OF THE INVENTION

Embodiments of the invention relate to compositions and methods for treating and/or preventing damage caused by ROS and/or inflammatory cytokines in mammals. More specifically, embodiments of the invention relate to the prevention and/or reduction of skeletal muscle damage and muscle pain through the administration of histamine and histamine agonists.

In some embodiments, the invention described herein relates to compositions and methods for treating and/or preventing disorders affecting muscle function, including, skeletal muscle damage and muscle pain, caused by reactive oxygen species in mammals, such as, humans. More specifically, the disclosure relates to the treatment and prevention of skeletal muscle damage through the administration of histamine and related compounds. In other embodiments, the invention relates to methods for reducing skeletal muscle damage and muscle pain caused by reactive oxygen species in mammals. In one embodiment, a method for inhibiting and reducing reactive oxygen species (ROS)-mediated oxidative damage to skeletal muscles of a subject is provided, comprising the step of administering a compound effective to inhibit the production or release of enzymatically-produced ROS to a subject suffering from a disorder caused or exacerbated by ROS-mediated oxidative damage. Although the compositions and methods are applicable to any disorders that affect skeletal muscular function, they are particularly relevant to the treatment of disorders selected from the group consisting of fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle.

Another embodiment relates to compositions and methods for treating a subject suffering from a disease state wherein reactive oxygen species (ROS)-mediated oxidative damage and/or inflammatory cytokine-mediated damage can occur. The methods can include the steps of comprises identifying a subject with a disorder affecting skeletal muscular function in which ROS and/or inflammatory cytokines cause damage and administering a compound effective to inhibit the production or release of ROS and/or inflammatory cytokines.

Subjects suffering from disorders affecting skeletal muscular function can be identified by methods known in the art, such as, for example, by blood and urine tests, computerized tomography (CT Scan), magnetic resonance imaging (MRI), ultrasound, electromyography (EMG), ultrasonography, neurologic examination, spinal tap (lumbar puncture), nerve conduction velocity (NCV), flexibility tests, range of motion tests, muscle strength tests, palpation of tender points, physical examination, and muscle biopsy.

Advantageously, the compound effective to inhibit the production or release of reactive oxygen species is histamine, a histamine receptor agonist, a histamine prodrug, a NADPH oxidase inhibitor, serotonin or a serotonin agonist. Optionally, the composition further includes an effective amount of a ROS scavenger. The ROS scavenger can be catalase, superoxide dismutase, glutathione peroxidase, or ascorbate peroxidase, or analogues thereof. The scavenger can also be vitamin A, vitamin E, or vitamin C. Alternatively, the ROS scavenger can be N-acetylcysteine.

Optionally, the method further includes the step of administering an effective amount of a ROS scavenger. Advantageously, the step of administering said ROS scavenger results in ROS scavenger-catalyzed decomposition of ROS. The scavenger can be catalase, glutathione peroxidase, ascorbate peroxidase, and superoxide dismutase, or analogues thereof. The scavenger can also be vitamin A, vitamin E, or vitamin C. Alternatively, the ROS scavenger can be N-acetylcysteine.

In one embodiment of the invention, methods and compositions for reducing skeletal muscle damage associated with fibromyalgia are provided. Skeletal muscle damage can include widespread muscle pain, muscle tenderness, muscle stiffness, muscle spasms, muscle twitching (fasciculation), numbness or tingling of the muscles, and muscle atrophy. The method includes administering to a subject in need thereof an effective amount of a compound effective to inhibit the production or release of ROS and/or inflammatory cytokines. Advantageously, the compound to inhibit the production or release of ROS and/or inflammatory cytokines includes histamine, histamine receptor agonists, NADPH oxidase inhibitors, serotonin and serotonin agonists. Optionally, the method can include a further step of administering an effective amount of a ROS scavenger. Preferably, the step of administering the ROS scavenger results in ROS scavenger-catalyzed decomposition of ROS. The scavenger can be catalase, glutathione peroxidase, ascorbate peroxidase, or superoxide dismutase, or analogues thereof. Additionally, the scavenger can be vitamin A, vitamin E, or vitamin C. Alternatively, the ROS scavenger can be N-acetylcysteine.

In another embodiment of the invention, methods and compositions for reducing skeletal muscle damage associated with amyotrophic lateral sclerosis (ALS) is provided. Skeletal muscle damage can include loss of muscle control or paralysis, muscle cramps or twitching (fasciculation), muscle spasms, loss of strength or coordination, muscle pain, muscle weakness, and muscle atrophy. The method includes administering to a subject in need thereof an effective amount of a compound effective to inhibit the production or release of ROS and/or inflammatory cytokines. Advantageously, the compound to inhibit the production or release of ROS and/or inflammatory cytokines includes histamine, histamine receptor agonists, NADPH oxidase inhibitors, serotonin and serotonin agonists. Optionally, the method can include a further step of administering an effective amount of a ROS scavenger. Preferably, the step of administering the ROS scavenger results in ROS scavenger-catalyzed decomposition of ROS. The scavenger can be catalase, glutathione peroxidase, ascorbate peroxidase, or superoxide dismutase, or analogues thereof. The scavenger can also be vitamin A, vitamin E, or vitamin C. Alternatively, the ROS scavenger can be N-acetylcysteine.

In still another embodiment of the invention, methods and compositions for reducing damage to skeletal muscles associated with multiple sclerosis (MS) is provided. Damage to skeletal muscles can include numbness, weakness, paralysis, pain, tingling, muscle twitching (fasciculation), muscle spasms, and muscle atrophy. The method includes administering to a subject in need thereof an effective amount of a compound effective to inhibit the production or release of ROS and/or inflammatory cytokines. Advantageously, the compound to inhibit the production or release of ROS and/or inflammatory cytokines includes histamine, histamine receptor agonists, NADPH oxidase inhibitors, serotonin and serotonin agonists. Optionally, the method can include a further step of administering an effective amount of a ROS scavenger. Preferably, the step of administering the ROS scavenger results in ROS scavenger-catalyzed decomposition of ROS. The scavenger can be catalase, glutathione peroxidase, ascorbate peroxidase, or superoxide dismutase, or analogues thereof. Additionally, the scavenger can be vitamin A, vitamin E, or vitamin C. Alternatively, the ROS scavenger can be N-acetylcysteine.

The compound can be administered in a single dose or in multiple doses, and can be administered at a rate of about 0.25 to about 1.0 mg/min. In some embodiments, the compound is administered in a dosage of about 0.2 mg to about 100 mg. In some embodiments, the administration of the compound is accomplished by a method selected from the group consisting of injection, intramuscular injection, intravenous injection, implantation infusion device, inhalation, and transdermal diffusion. In some embodiments, the compound is administered over a time period of between about 1 and 30 minutes. In some embodiments, the compound is administered over a time period of between about 1 and 30 days. In some embodiments, the compound is administered orally. The compound can be in a form selected from the group consisting of capsules, tablets, granules, sprays, and syrups. In some embodiments, the compound is administered in a sustained release formulation.

Some embodiments further include the step of administering an additional compound selected from the group consisting of an anti-inflammatory drug, a pain reliever, and a muscle relaxant.

In some embodiments, the use of a compound effective to reduce the amount of ROS in the preparation of medicament for treating a disorder affecting muscle function is disclosed. In other embodiments, the use of a compound effective to reduce the amount of ROS in the preparation of medicament for treating muscle pain associated with reactive oxygen species (ROS)-mediated oxidative damage is disclosed. In other embodiments, the use of a compound effective to reduce the amount of ROS in the preparation of medicament for inhibiting reactive oxygen species (ROS)-mediated oxidative damage to skeletal muscles of a subject are disclosed.

### DETAILED DESCRIPTION

The disclosure below relates to compositions and methods for preventing and reducing damage to skeletal muscles caused by reactive oxygen species (ROS) and/or inflammatory cytokines.

Skeletal muscles play an essential role in movement and well being. Disorders affecting muscle function typically have serious consequences for the person afflicted, ranging from morbidity to mortality. Examples of disorders affecting skeletal muscle function include, for example, but not limited to: fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle.

Recent work has indicated that these and other disorders affecting muscle function may be exacerbated by ROS and/or inflammatory cytokines. ROS and/or inflammatory cytokines can have direct effects on various cells within the muscles leading to apoptotic myonuclear elimination. Another possible mechanism by which these molecules can damage muscle cells and tissue may be related to the role of ROS in ischemia/reperfusion, aging, muscle injury, inflammation, and exercise. For example, ROS produced during exercise may effectively accelerate muscle damage.

One embodiment of the invention relates to compositions and methods for preventing and/or reducing muscle damage and/or muscle pain caused by ROS and/or inflammatory cytokines. The compositions and methods of the invention contemplate reducing ROS-mediated damage and/or pain or inflammatory cytokine-mediated damage and/or pain by reducing the production and release of ROS and/or inflammatory cytokines.

A variety of reactive oxygen metabolites are produced in the monovalent pathway of oxygen reduction. These ROS are enzymatically produced by phagocytes such as monocytes and polymorphonuclear neutrophils (PMNs) and frequently released in a respiratory burst. Neutrophils also produce ROS constitutively. The constitutive production may contribute to ROS-mediated cellular damage. Hydrogen peroxide and other ROS play an important role in a host's immunological defenses. Nevertheless, ROS produced in excessive amounts or at inappropriate times or locations, act to damage a host's cells and tissues, and thus can be detrimental to the host.

The effects of ROS production are many faceted. ROS are known to cause apoptosis in NK cells. ROS are also known to cause anergy and/or apoptosis in T-cells. The mechanisms by which ROS cause these effects are not yet fully understood. Nevertheless, some commentators believe that ROS cause cell death by disrupting cellular membranes and by changing the pH of cellular pathways critical for cell survival and also by direct damaging effects on DNA.

Additionally, phagocytes that undergo a respiratory burst, and produce and release large quantities of ROS also produce and release inflammatory cytokines such as tumor necrosis factor (TNF) and interleukin-1 (IL-1). An example of inflammatory cytokine-mediated cell damage is found in the Shwartzman Reaction, where neutrophil mediated cell damage is thought to be activated by TNF and IL-1. Imarnura S, et al., “Involvement of tumor necrosis factor-alpha, interleukin-1 beta, interleukin-8, and interleukin-1 receptor antagonist in acute lung injury caused by local Shwartzman reaction,” Pathol. Int. 47(1):16-24 (1997). This ROS and cytokine release augments the cell damage inflicted by a variety of sources as these potent chemical compounds are disseminated throughout the body. Although released as a defensive measure by the cells of the immune system, the ROS result in ROS-mediated cell damage and the inflammatory cytokines cause a rapid deterioration of the patient, resulting often in death.

Compounds that reduce or inhibit the amount of ROS and/or inflammatory cytokines produced or released by sources within a subject can facilitate the treatment and recovery of individuals suffering from disorders affecting muscle function. The conditions contemplated as treatable under the embodiments of the invention result from a disparate number of etiological causes. Nevertheless, they share a common feature in that their pathological conditions are either caused or exacerbated by enzymatically produced, ROS-mediated oxidative damage or inflammatory cytokine-mediated damage, caused by inappropriate and harmful concentrations of ROS and/or inflammatory cytokines. Thus, the administration of compounds that inhibit the production or release of ROS and/or inflammatory cytokines, or compounds that scavenge ROS, alone or in combination with other beneficial compounds, provides an effective treatment for a variety of disorders affecting muscle function.

Embodiments of the invention contemplate compounds and methods that are efficacious in treating a variety of conditions wherein ROS and/or inflammatory cytokines play an active, detrimental role in the pathological state of the disease. Such conditions include, but are not limited to: fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle.

The compounds which reduce the amount of ROS and/or inflammatory cytokines produced and released in an individual and the methods disclosed below are directed to the reduction and prevention of ROS-mediated damage or inflammatory cytokine-mediated damage of skeletal muscles and/or muscle pain. In preferred embodiments, various histamine and histamine-related compounds are used to achieve a beneficial reduction or inhibition of enzymatic ROS and/or inflammatory cytokine production and release or the net concentration thereof. Histamine and histamine-related compounds include, or specifically exclude, any of, histamine, the dihydrochloride salt form of histamine (histamine dihydrochloride), histamine diphosphate, other histamine salts, histamine esters, histamine prodrugs, and histamine receptor agonists. Other ROS and/or inflammatory cytokine production and release inhibitory compounds such as, NADPH oxidase inhibitors like diphenyleneiodonium, can also be used with the disclosed methods, as can serotonin and 5HT-receptor agonists.

The administration of compounds that induce the release of endogenous histamine from a patient's own tissue stores is also included within the scope of the present disclosure. Such compounds include, for example, without limitation, IL-3, retinoids, and allergens.

The compositions and methods disclosed herein also encompass the administration of a variety of ROS scavengers. Known scavengers of ROS include the enzymes catalase, superoxide dismutase (SOD), glutathione peroxidase, ascorbate peroxidase, or analogues thereof. Additionally, vitamins A, E, and C are known to have scavenger activity. Alternatively, the ROS scavenger can be N-acetylcysteine. Minerals such as selenium and manganese can also be efficacious in combating ROS-mediated damage. The scope of the methods disclosed herein includes the administration of the compounds listed and those compounds with similar ROS inhibitor activity. The compositions and methods disclosed herein also provide an effective means for preventing and/or inhibiting the release of enzymatically generated ROS in excessive amounts or at inappropriate times or locations.

Compounds and methods for treating disorders affecting muscle function that are complicated by the detrimental release of ROS and/or inflammatory cytokines within a host or subject are provided. Muscles are responsible for many essential functions in the body. The impairment of these functions by disorders affecting these functions can lead to very serious consequences. Muscle damage has been linked to a number of sources. For example, it may be caused by infections with bacteria, parasites, yeasts, or viruses.

Examples of environmental and industrial toxins which cause damage to skeletal muscles include, without limitation, cigarette smoke, caffeine, alcohol, chemical solvents, detergents, petroleum products, radiation, pesticides, fungicides, herbicides, industrial pollutants, and toxic metal compounds, including lead, aluminum, manganese and mercury, such as that from dental amalgams and contaminated fish. Toxins also include many common drugs, such as synthetic hormones. Damage to skeletal muscle results, at least in part, by the detrimental release of ROS and/or inflammatory cytokines within a host or subject in response to such insults. Accordingly, compositions and methods for treating damage to skeletal muscle caused by exposure to toxic substances are provided. Specifically, the administration of a ROS and/or inflammatory cytokine production and release inhibiting compound is useful for the reduction in trauma to muscle cells and tissues following exposure to industrial and/or environmental toxins.

The administration of a ROS and/or inflammatory cytokines inhibitor or ROS scavenger is likewise useful for ameliorating damage to muscle tissue caused or exacerbated by infection with yeasts, bacteria, viruses, and parasites. Accordingly, in one embodiment, compounds and methods for minimizing damage to muscle tissue associated with bacterial, viral, yeast, or parasite infections are provided. ROS and/or inflammatory cytokine production and release inhibiting compounds are administered alone or in combination with an antibiotic. As used herein, the term “antibiotic” includes any antibacterial or antifungal compound.

When administered in combination with antibiotics, the ROS and/or inflammatory cytokine production and release inhibiting compound can be administered separately or as a single formulation with the antibiotic. If administered separately, the ROS and/or inflammatory cytokine production and release inhibiting compound should be given in a temporally proximate manner such that the amelioration of damage to muscle tissue is enhanced. In one embodiment, the ROS and/or inflammatory cytokine production and release inhibiting compound and antibiotic are given within one week of each other. In another embodiment, the ROS and/or inflammatory cytokine production and release inhibiting compound and antibiotic are given within twenty-four hours of each other. In yet another embodiment, the ROS and/or inflammatory cytokine production and release inhibiting compound and antibiotic are given within one hour of each other. The administration can be by either local or systemic delivery. Other methods of administration are also suitable, such as oral administration.

The administration of the disclosed compounds can be alone or in combination with other compounds effective for treating various disorders affecting skeletal muscle function. For example, histamine alone can be used to treat a patient suffering from such disorders. Further, the disclosed methods and compounds can be used in combination with standard treatment regimes for disorders affecting muscle function, which usually comprise administration of anti-inflammatory drugs, including nonsteroidal anti-inflammatory drugs (NSAIDs), such as aspirin, ibuprofen, and acetaminophen; pain relievers, including non-narcotic pain relievers, such as tramadol; lidocaine injections; muscle relaxants, such as cyclobenzaprine, tizanidine, amantadine, and modafinil; antidepressants, such as amitriptyline, notriptyline, trazodone, doxepin, citalopram, fluoxetine, sertraline, and paroxetine; sleeping pills, such as zolpidem tartrate; benzodiazepines; riluzole; beta interferons; glatiramer; mitoxantrone; and corticosteroids. For example, in the case of fibromyalgia, individuals presenting with the disorder are administered an effective dose of a ROS and/or inflammatory cytokine inhibiting compound or scavenger along with standard nonsteroidal anti-inflammatory drug (NSAID) protocols. In the case of multiple sclerosis (MS), a subject can be administered beta interferon concurrently with the administration of a ROS and/or inflammatory cytokine inhibiting compound or scavenger to minimize the loss of muscle function. In the case of amyotrophic lateral sclerosis (ALS), a subject can be administered an effective dose of a ROS and/or inflammatory cytokine inhibiting compound or scavenger along with riluzole to minimize muscle damage.

The administration of the ROS and/or inflammatory cytokine inhibiting or ROS scavenging compounds can be by any of a number of methods well known to those of skill in the art. For oral administration, the ROS and/or inflammatory cytokine inhibiting or scavenging compounds can be incorporated into a tablet, aqueous or oil suspension, dispersible powder or granule, microbead, emulsion, hard or soft capsule, syrup or elixir. The compositions can be prepared according to any method known in the art for the manufacture of pharmaceutically acceptable compositions and such compositions can contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets can be uncoated or can be coated using known techniques to delay disintegration and absorption in the gastrointestinal tract and thereby provide a sustained action over a longer period of time. For example, a time delay material such as glyceryl monostearate or glyceryl stearate alone or with a wax can be employed.

In other embodiments, tablets, capsules or microbeads containing the active ingredient are coated with an enteric coating which prevents dissolution in the acidic environment of the stomach. Instead, this coating dissolves in the small intestine at a more neutral pH. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, DC, 1998, the entire contents of which are hereby incorporated by reference.

Formulations for oral use can also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions can contain the ROS and/or inflammatory cytokine inhibiting or ROS scavenging compounds in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions can be formulated by suspending the active ingredient in a vegetable oil, such as arachis oil, olive oil, sesame oil or coconut oil, or in a mineral oil such as liquid paraffin. The oil suspension can contain a thickening agent, such as beeswax, hard paraffin or cetyl alcohol. Sweetening agents, such as those set forth above, and flavoring agents can be added to provide a palatable oral preparation. These compositions can be preserved by an added antioxidant such as ascorbic acid. Dispersible powders and granules of the compounds, suitable for preparation of an aqueous suspension by the addition of water, provide the active ingredient in admixture with a dispersing or wetting agent, a suspending agent, and one or more preservatives. Additional excipients, for example sweetening, flavoring and coloring agents, can also be present.

Syrups and elixirs can be formulated with sweetening agents, such as glycerol, sorbitol or sucrose. Such formulations can also contain a demulcent, a preservative, a flavoring or a coloring agent.

Administration of the ROS and/or inflammatory cytokine inhibiting or ROS scavenging compounds can also be accomplished via parenteral delivery, for example, but not limited to, through subcutaneous, intravenous, intraperitoneal, or intramuscular injection. The compounds can be administered in an aqueous solution with or without a surfactant such as hydroxypropyl cellulose. Dispersions are also contemplated such as those utilizing glycerol, liquid polyethylene glycols, and oils. Injectable preparations can include sterile aqueous solutions or dispersions and powders that can be diluted or suspended in a sterile environment prior to use. Carriers such as solvents or dispersion media contain water, ethanol polyols, vegetable oils and the like can also be added to the disclosed compounds. Coatings such as lecithins and surfactants can be used to maintain the proper fluidity of the composition. Isotonic agents such as sugars or sodium chloride can be added, as well as products intended to delay absorption of the active compounds such as aluminum monostearate and gelatin. Sterile injectable solutions are prepared according to methods well known to those of skill in the art and can be filtered prior to storage and/or use. Sterile powders can be vacuum or freeze dried from a solution or suspension. Sustained or controlled release preparations and formulations can also be used with the disclosed methods. Typically the materials used with the disclosed methods and compositions are pharmaceutically acceptable and substantially non-toxic in the amounts employed.

The disclosed compounds can also be administered by inhalation. In this administration route, histamine, for example, can be dissolved in water or some other pharmaceutically acceptable carrier liquid for inhalation, or provided as a dry powder, and then introduced into a gas or powder that is then inhaled by the patient in an appropriate volume so as to provide that patient with a measured amount of histamine. Examples of the administration of a therapeutic composition via inhalation are described in U.S. Pat. Nos. 6,418,926; 6,387,394; 6,298,847; 6,182,655; 6,132,394; and 6,123,936, which are hereby incorporated by reference.

Infusion devices can be used to deliver the disclosed compounds. Suitable devices include syringe pumps, auto injector systems, implantable pumps, implantable devices, and minipumps. Exemplary devices include the Ambulatory Infusion Pump Drive, Model 30, available from Microject Corp., Salt Lake City, Utah, and the Baxa Syringe Infuser, available from Baxa Corporation, Englewood, Colo. Any device capable of delivering the disclosed compounds in accordance with the methods disclosed herein can be used.

Suitable infusion devices preferably have an effective amount of histamine, histamine agonist, histamine salt, histamine prodrug, NADPH-oxidase inhibitor, histamine dihydrochloride, histamine phosphate, serotonin, a 5HT agonist, a histamine receptor agonist, histamine receptor binding mimic, or a substance which induces the release of an effective therapeutic amount of endogenous histamine, contained therein. The device can be pre-loaded with the desired substance during manufacture, or the device can be filled with the substance just prior to use. Pre-filled infusion pumps and syringe pumps are well known to those of skill in the art. The active substance can be part of a formulation which includes a controlled release carrier, if desired. A controller is used with the device to control the rate of administration and the amount of substance to be administered. The controller can be integral with the device or it can be a separate entity. It can be pre-set during manufacture, or set by the user just prior to use. Such controllers and their use with infusion devices are well known to those of skill in the art.

Controlled release vehicles are well known to those of skill in the pharmaceutical sciences. The technology and products in this art are variably referred to as controlled release, sustained release, prolonged action, depot, repository, delayed action, retarded release and timed release; the words “controlled release” as used herein is intended to incorporate each of the foregoing technologies.

Numerous controlled release vehicles are known, including biodegradable or bioerodable polymers such as polylactic acid, polyglycolic acid, and regenerated collagen. Known controlled release drug delivery devices include creams, lotions, tablets, capsules, gels, microspheres, liposomes, ocular inserts, minipumps, and other infusion devices such as pumps and syringes. Implantable or injectable polymer matrices, and transdermal formulations, from which active ingredients are slowly released are also well known and can be used in the disclosed methods.

In one embodiment, the disclosed compounds are administered through a topical delivery system. The controlled release components described above can be used as the means to deliver the disclosed compounds. A suitable topical delivery system comprises the disclosed compounds in concentrations taught herein, a solvent, an emulsifier, a pharmaceutically acceptable carrier material, penetration enhancing compounds, and preservatives. Examples of topically applied compositions include U.S. Pat. Nos. 5,716,610 and 5,804,203, which are hereby incorporated by reference. The compositions can further include components adapted to improve the stability or effectiveness of the applied formulation, such as preservatives, antioxidants, skin penetration enhancers and sustained release materials. Examples of such components are described in the following reference works hereby incorporated by reference: Martindale—The Extra Pharmacopoeia (Pharmaceutical Press, London 1993) and Martin (ed.), Remington's Pharmaceutical Sciences.

Controlled release preparations can be achieved by the use of polymers to complex or absorb the ROS and/or inflammatory cytokine inhibiting or ROS scavenging compound. The controlled delivery can be exercised by selecting appropriate macromolecules such as polyesters, polyamino acids, polyvinylpyrrolidone, ethylenevinyl acetate, methylcellulose, carboxymethylcellulose, and protamine sulfate, and the concentration of these macromolecules as well as the methods of incorporation are selected in order to control release of active compound.

Hydrogels, wherein the ROS and/or inflammatory cytokine inhibiting or ROS scavenging compound is dissolved in an aqueous constituent to gradually release over time, can be prepared by copolymerization of hydrophilic mono-olefinic monomers such as ethylene glycol methacrylate. Matrix devices, wherein the ROS and/or inflammatory cytokine inhibiting or ROS scavenging compound is dispersed in a matrix of carrier material, can be used. The carrier can be porous, non-porous, solid, semi-solid, permeable or impermeable. Alternatively, a device comprising a central reservoir of the ROS and/or inflammatory cytokine inhibiting or ROS scavenging compound surrounded by a rate controlling membrane can be used to control the release of the ROS and/or inflammatory cytokine inhibiting or ROS scavenging compound. Rate controlling membranes include ethylene-vinyl acetate copolymer or butylene terephthalate/polytetramethylene ether terephthalate. Use of silicon rubber depots are also contemplated.

Controlled release oral formulations are also well known. In one embodiment, the active compound is incorporated into a soluble or erodible matrix, such as a pill or a lozenge. Such formulations are well known in the art. An example of a lozenge used to administer pharmaceutically active compounds is U.S. Pat. No. 5,662,920, which is hereby incorporated by reference. In another example, the oral formulations can be a liquid used for sublingual administration. An example of pharmaceutical compositions for liquid sublingual administration of the disclosed compounds are taught in U.S. Pat. No. 5,284,657, which is hereby incorporated by reference. These liquid compositions can also be in the form a gel or a paste. Hydrophilic gums, such as hydroxymethylcellulose, are commonly used. A lubricating agent such as magnesium stearate, stearic acid, or calcium stearate can be used to aid in the tableting process.

For the purpose of parenteral administration, ROS and/or inflammatory cytokine inhibiting or ROS scavenging compounds can be combined with distilled water, preferably buffered to an appropriate pH and having appropriate (e.g., isotonic) salt concentrations. The compounds can also be provided as a liquid or as a powder that is reconstituted before use. They can be provided as prepackaged vials, syringes, or injector systems.

The disclosed compounds, such as histamine, can also be provided in septum-sealed vials in volumes ranging from about 0.5 to about 100 ml for administration to an individual. The vials are preferably sterile. The vials can optionally contain an isotonic carrier medium and/or a preservative. Any desired amount of histamine or other ROS and/or inflammatory cytokine inhibitory compound can be used to give a desired final concentration. In a preferred embodiment, the ROS and/or inflammatory cytokine inhibiting or ROS scavenging concentration is between about 0.01 mg/ml and about 100 mg/ml. More preferably, the ROS and/or inflammatory cytokine inhibiting or ROS scavenging compound concentration is between about 0.1 and about 50 mg/ml. Most preferably, the ROS and/or inflammatory cytokine inhibiting or ROS scavenging compound concentration is between about 1 mg/ml and about 10 mg/ml. At the lower end of the volume range, it is preferred that individual doses are administered, while at the higher end it is preferred that multiple doses are administered.

In another embodiment, transdermal patches, steady state reservoirs sandwiched between an impervious backing and a membrane face, and transdermal formulations, can also be used to deliver ROS and/or inflammatory cytokine inhibiting or ROS scavenging compounds. Transdermal administration systems are well known in the art. Occlusive transdermal patches for the administration of an active agent to the skin or mucosa are described in U.S. Pat. Nos. 4,573,996, 4,597,961 and 4,839,174, which are hereby incorporated by reference. One type of transdermal patch is a polymer matrix in which the active agent is dissolved in a polymer matrix through which the active ingredient diffuses to the skin. Such transdermal patches are disclosed in U.S. Pat. Nos. 4,839,174, 4,908,213 and 4,943,435, which are hereby incorporated by reference. In one embodiment, the steady state reservoir carries doses of histamine or other ROS and/or inflammatory cytokine production and release inhibitory or scavenging compounds in doses from about 0.2 to about 20 mg per day.

Present transdermal patch systems are designed to deliver smaller doses over longer periods of time, up to days and weeks. A preferred delivery system for the disclosed compounds would specifically deliver an effective dose of, for example, histamine, in a range of between about 1 and about 60 minutes, depending upon the dose, with a preferred dose being delivered within about 20 to 30 minutes. These patches allow rapid and controlled delivery of a compound which inhibits or scavenges ROS and/or inflammatory cytokines. A rate-controlling outer microporous membrane, or micropockets of the disclosed compounds dispersed throughout a silicone polymer matrix, can be used to control the release rate. Such rate-controlling means are described in U.S. Pat. No. 5,676,969, which is hereby incorporated by reference. In another embodiment, the histamine or other ROS and/or inflammatory cytokine inhibiting or ROS scavenging compound is released from the patch into the skin of the patient in about 20 to 30 minutes or less. In one embodiment, the compound is released from the patch at a rate of between about 0.025 mg to about 0.5 mg per minute for a dose of between about 0.2 mg and about 20 mg per patch.

These transdermal patches and formulations can be used with or without use of a penetration enhancer such as dimethylsulfoxide (DMSO), combinations of sucrose fatty acid esters with a sulfoxide or phosphoric oxide, or eugenol. The use of electrolytic transdermal patches is also within the scope of the methods disclosed herein. Electrolytic transdermal patches are described in U.S. Pat. Nos. 5,474,527, 5,336,168, and 5,328,454, the entire contents of which are hereby incorporated by reference.

In another embodiment, transmucosal patches can be used to administer the disclosed compounds. An example of such a patch is found in U.S. Pat. No. 5,122,127, which is hereby incorporated by reference. The described patch comprises a housing capable of enclosing a quantity of therapeutic agent where the housing is capable of adhering to mucosal tissues, for example, in the mouth. A drug surface area of the device is present for contacting the mucosal tissues of the host. The device is designed to deliver the drug in proportion to the size of the drug/mucosa interface. Accordingly, drug delivery rates can be adjusted by altering the size of the contact area.

The housing is preferably constructed of a material which is nontoxic, chemically stable, and non-reactive with the disclosed compounds. Possible construction materials include: polyethylene, polyolefins, polyamides, polycarbonates, vinyl polymers, and other similar materials known in the art. The housing can contain means for maintaining the housing positioned against the mucosal membrane. The housing can contain a steady state reservoir positioned to be in fluid contact with mucosal tissue.

Steady state reservoirs for use with the disclosed compounds deliver a suitable dose of those compounds over a predetermined period of time. Compositions and methods of manufacturing compositions capable of absorption through the mucosal tissues are taught in U.S. Pat. No. 5,288,497, which is hereby incorporated by reference. One of skill in the art could readily include the disclosed compounds and related compositions.

The steady state reservoirs for use with the disclosed compounds are composed of compounds known in the art to control the rate of drug release. In one embodiment, the transmucosal patch delivers a dose of a ROS and/or inflammatory cytokine inhibiting or ROS scavenging compound over a period of time from about 1 to about 60 minutes. The steady state reservoir contained within the housing carries doses of histamine or other ROS and/or inflammatory cytokine production and release inhibitory compounds in doses from about 0.1 to about 20 mg per patch. Transdermal patches that can be worn for several days and that release the disclosed compounds over that period of time are also contemplated. The reservoirs can also contain permeation or penetration enhancers, as discussed above, to improve the permeability of the disclosed compounds across the mucosal tissue.

Another method to control the release of the disclosed compounds is to incorporate the ROS and/or inflammatory cytokine inhibiting or ROS scavenging compound into particles of a polymeric material such as polyesters, polyamino acids, hydrogels, poly lactic acid, or ethylene vinylacetate copolymers.

Alternatively, instead of incorporating the ROS and/or inflammatory cytokine inhibiting or ROS scavenging compounds into these polymeric particles, the disclosed compounds can be entrapped in microcapsules prepared, for example, by coacervation techniques, or by interfacial polymerization, for example hydroxymethylcellulose or gelatin-microcapsules, respectively, or in colloidal drug delivery systems, for example, liposomes, albumin microspheres, microemulsions, nanoparticles, and nanocapsules, or in macroemulsions. Such technology is well known to those of ordinary skill in pharmaceutical sciences.

Preferably, the compounds that inhibit ROS and/or inflammatory cytokines are injected, infused, or released into the patient at a rate of from about 0.025 mg/min to about 1.0 mg/min. A rate of about 0.1 mg/min is preferred. The disclosed compounds are preferably administered over a period of time ranging from about 1 minute to about 60 minutes, with about 20 minutes being preferred, such that the total daily adult dose of ROS and/or inflammatory cytokine inhibiting or ROS scavenging compound ranges from between about 0.1 mg to about 200 mg, with about 0.2 mg to about 20 mg being preferred. Administration of the compounds over longer periods of time, i.e., longer than about 30 minutes, has been found to result in decreased or lack of efficacy, while rapid administration over less than 1 to 3 minutes can cause more pronounced and serious side effects, which include anaphylaxis, heart failure, bronchospasm, pronounced flushing, discomfort, increased heart rate and respiratory rate, hypotension, and severe headache.

In another embodiment, a ROS and/or inflammatory cytokine inhibiting or ROS scavenging compound at approximately 3 μg/kg body weight to about 10 mg/kg body weight, in a pharmaceutically acceptable form can be administered. ROS scavenging compounds can also be administered in combination with the ROS and/or inflammatory cytokine production and release inhibitory compounds described above.

The treatment can also include periodically boosting patient blood ROS and/or inflammatory cytokine inhibiting or ROS scavenging compound levels by administering additional compound in amounts ranging from about 3 μg/kg body weight to about 10 mg/kg body weight, one to four times per day over a period of one to two weeks at regular intervals, such as daily, bi-weekly, or weekly in order to establish blood levels of ROS and/or inflammatory cytokine inhibiting or ROS scavenging compound at a beneficial concentration such that ROS and/or inflammatory cytokine production and release is inhibited. The administration can be by any of the means described above. The treatment is continued until the causes of the patient's underlying disease state are controlled or eliminated.

Administration of each dose of ROS and/or inflammatory cytokine inhibiting or ROS scavenging compound can occur from once a day to up to about four times a day, with twice a day being preferred. Administration can be subcutaneous, intraperitoneal, intravenous, intramuscular, intraocular, oral, transdermal, intranasal, or rectal and can utilize direct hypodermic or other injection or infusion means, or can be mediated by a controlled release mechanism of the type disclosed above. Any controlled release vehicle or infusion device capable of administering a therapeutically effective amount of the disclosed compounds over a period of time ranging from about 1 minute to about 60 minutes can be used. In one embodiment, intranasal delivery is accomplished by using a solution of ROS inhibiting or scavenging compound in an atomizer or nebulizer to produce a fine mist which is introduced into the nostrils. For rectal delivery, ROS and/or inflammatory cytokine inhibiting or scavenging compound is formulated into a suppository using methods well known in the art.

In another embodiment, the ROS and/or inflammatory cytokine inhibiting or ROS scavenging compound can be administered orally. When administered orally, the compound can be administered in capsule, tablet, granule, spray, syrup, or other such form. In one embodiment, the composition can be formulated as a tablet comprising between about 10 mg to about 2 grams of active ingredient. For example, such a tablet can include 10, 20, 50, 100, 150 200, 500, 1,000, or 2,000 milligrams of ROS and/or inflammatory cytokine inhibiting or ROS scavenging compound. Preferably, the amount of ROS inhibiting or scavenging compound in a tablet is about 100 mg. In some embodiments, the composition includes histamine protectors such as diamine oxidase inhibitors, monoamine oxidase inhibitors and n-methyl transferases.

Compounds that scavenge ROS can be administered in an amount of from about 0.2 mg/day to about 20 mg/day; more preferably, the amount is from about 0.5 mg/day to about 8 mg/day; and even more preferably, the amount is from about 1 mg/day to about 5 mg/day. In each case, the dose depends on the activity of the administered compound. The foregoing doses are appropriate for the enzymes listed above that include catalase, superoxide dismutase (SOD), glutathione peroxidase and ascorbate peroxidase. Appropriate doses for any particular host can be readily determined by empirical techniques well known to those of ordinary skill in the art.

Non-enzymatic ROS scavengers can be administered in amounts empirically determined by one of ordinary skill in the art. For example, vitamins A and E can be administered in doses from about 1 to about 5000 IU per day. Vitamin C can be administered in doses from about 1 μg to about 10 g per day. Minerals such as selenium and manganese can be administered in amounts from about 1 picogram to about 1 milligram per day. These compounds can also be administered as a protective or preventive treatment for ROS-mediated disease states.

As noted above, in addition to histamine, histamine dihydrochloride, histamine phosphate, other histamine salts, histamine esters, histamine congeners, histamine prodrugs, and H₂ receptor agonists, the use of serotonin, 5HT agonists, and compounds which induce release of histamine from the patient's own tissues are all included within the disclosed compounds and methods. Retinoic acid, other retinoids such as 9-cis-retinoic acid and all-trans-retinoic acid, IL-3 and ingestible allergens are compounds that are known to induce the release of endogenous histamine. These compounds can be administered to the patient by the means described above, including oral, intravenous, intramuscular, subcutaneous, and other approved routes. The rate of administration preferably results in a release of endogenous histamine resulting in a blood plasma level of histamine of about 20 nmol/dl.

Administration of each dose of a compound which induces histamine release can occur from once per day to up to about four times a day, with twice per day being preferred. Administration can be subcutaneous, intravenous, intramuscular, intraocular, oral, or transdermal, and can incorporate a controlled release mechanism of the type disclosed above. Any controlled release vehicle capable of administering a therapeutically effective amount of a compound which induces histamine release over a period of time ranging from about one to about thirty minutes can be used. Additionally, the compounds, compositions, and formulations of embodiments of the invention can be administered as needed to ease the pain or discomfort of the subject.

The following examples teach various methods for treating disorders affecting muscle function with the disclosed ROS and/or inflammatory cytokine production and release inhibiting compounds. These examples are illustrative only and are not intended to limit the scope of the claims. The treatment methods described below can be optimized using empirical techniques well known to those of ordinary skill in the art. Moreover, artisans of ordinary skill would be able to use the teachings described in the following examples to practice the full scope of the claims. Although it is stated in the examples that the administration of a ROS and/or inflammatory cytokine inhibiting or ROS scavenging compound can be given in a single dose, it is obvious that the compounds can be distributed over longer periods of time. Moreover, the daily dose can be administered as a single dose or it can be divided into several doses.

### EXAMPLES

### Example 1

### Inhibition of Muscle Pain Associated with Fibromyalgia

A female patient, age 60, suffering from Acute Myelogenous Leukemia (AML) and fibromyalgia received treatment with histamine dihydrochloride and IL-2. Histamine dihydrochloride was administered subcutaneously two times per day at a dosage of 0.5 mg per injection for a period of 21 consecutive days.

The above 21-day cycle was repeated for 18 months with 21-day intermissions between cycles.

The patient's symptoms disappeared while on treatment and returned during intermissions.

### Example 2

### Inhibition of Muscle Pain Associated with Amyotrophic Lateral Sclerosis

A male patient, age 65, with amyotrophic lateral sclerosis received treatment with histamine dihydrochloride. Histamine dihydrochloride was administered orally at a dosage of 1 mg/day for a period of six months.

The progression of ALS was arrested.

### Example 3

### Inhibition of Muscle Pain

Subjects suffering from muscle pain exacerbated by ROS and/or inflammatory cytokines are identified. The subjects are asked to rate their pain on a scale of 1 to 10, with 1 indicating no pain or mild pain and 10 indicating severe pain. The subjects are then separated into 11 groups of 10 subjects each. Subjects in Groups 1 through 10 are administered an effective dose of histamine, histamine agonists, histamine salts, histamine prodrugs, NADPH-oxidase inhibitors, histamine dihydrochloride, histamine phosphate, serotonin, 5HT agonists, histamine receptor agonists, or histamine receptor binding mimics, respectively. Subjects in Group 11 are administered a placebo. After several weeks of treatment, subjects are again asked to rate their pain using the scale above. Almost all subjects in Groups 1 through 10 report a reduction in muscle pain. Subjects in Group 11 report no change or an increase in muscle pain.

### Example 4

### Treatment of Fibromyalgia

Individuals suffering from fibromyalgia are identified. The individuals are asked to rate their pain on a scale of 1 to 10, with 1 indicating no pain or mild pain and 10 indicating severe pain. The individuals are then divided into 5 groups of 25 individuals each. Individuals in Groups 1 through 4 are intravenously administered 0.5 mg, 1 mg, 5 mg, and 20 mg of histamine prodrugs, respectively. Individuals in Group 5 are administered a placebo. The histamine prodrugs or placebos are administered in conjunction with standard non-steroid anti-inflammatory drug (NSAID) treatment regimes. Individuals are again asked to rate their pain using the scale above. Almost all individuals in Groups 1 through 4 report a decrease in pain in a dose responsive manner. Individuals in Group 5 report no change or an increase in pain.

### Example 5

### Treatment of Multiple Sclerosis

Individuals suffering from multiple sclerosis are identified. The individuals are asked to fill out a questionnaire regarding commonly experienced symptoms experienced by MS patients as well as the rate and severity of each symptom. The individuals are then separated into 7 groups of 20 individuals. Individuals in Groups 1 through 6 are orally administered 10 mg, 30 mg, 50 mg, 100 mg, 200 mg, and 1,000 mg of histamine, respectively. Individuals in Group 7 are administered a placebo. Following the course of treatment, the individuals are again asked to fill out the same questionnaire. Groups 1 through 6 report a decrease in both the rate and severity of symptoms associated with muscle damage. Individuals in Group 7 report no change or an increase in the rate and/or severity of symptoms.

### Example 6

### Treatment of Amyotrophic Lateral Sclerosis

Individuals suffering from amyotrophic lateral sclerosis are identified. The individuals undergo a muscle strength test. The individuals are then intramuscularly administered 10 mg of NADPH-oxidase inhibitors or a placebo. The individuals are again tested for muscle strength. The rate of loss of muscle strength is minimized for individuals who received the NADPH-oxidase inhibitor as compared to individuals who received a placebo.

The foregoing description details certain embodiments of the invention. It will be appreciated, however, that no matter how detailed the foregoing appears in text, the invention can be practiced in many ways. As is also stated above, it should be noted that the use of particular terminology when describing certain features or embodiments of the invention should not be taken to imply that the terminology is being re-defined herein to be restricted to including any specific characteristics of the features or embodiments of the invention with which that terminology is associated. The scope of the invention should therefore be construed in accordance with the appended claims and any equivalents thereof.

What is claimed is:

1. A method for treating a disorder affecting muscle function comprising:
identifying a subject with a disorder affecting muscle function; and
administering an effective amount of a compound effective to inhibit the production or release of ROS to said subject.

2. The method of claim 1, wherein said disorder is selected from the group consisting fibromyalgia, multiple sclerosis (MS), amyotrophic lateral sclerosis (ALS or Lou Gehrig's disease), primary lateral sclerosis, spinal muscular atrophy, benign focal amyotrophy, muscular dystrophies, myopathies, myositis, lupus, and metabolic diseases of the muscle.

3. The method of claim 1, wherein said disorder is fibromyalgia.

4. The method of claim 1, wherein said disorder is amyotrophic lateral sclerosis (ALS).

5. The method of claim 1, wherein said disorder is multiple sclerosis (MS).

6. The method of claim 1, wherein said compound effective to inhibit the production or release of ROS is selected from the group consisting of a compound effective to inhibit the production or release of ROS, a ROS scavenger, and combinations thereof.

7. The method of claim 6, wherein said compound effective to inhibit the production or release of ROS is selected from the group consisting of histamine, histamine receptor agonists, histamine salts, histamine prodrugs, NADPH-oxidase inhibitors, serotonin, serotonin (5HT) receptor agonists, and substances which induce the release of an effective therapeutic amount of endogenous histamine.

8. The method of claim 6, wherein the step of administering said ROS scavenger results in ROS scavenger-catalyzed decomposition of ROS.

9. The method of claim 6, wherein the scavenger is selected from the group consisting of catalase, glutathione peroxidase, ascorbate peroxidase, and superoxide dismutase, or analogues thereof.

10. The method of claim 6, wherein the scavenger is N- acetylcysteine.

11. The method of claim 6, wherein the scavenger is selected from the group consisting of vitamin A, vitamin E, and vitamin C.

12. The method of claim 1, wherein said compound is administered in a single dose.

13. The method of claim 1, wherein said compound is administered in multiple doses.

14. The method of claim 1, wherein said compound is administered at a rate of about 0.025 to about 1.0 mg/min.

15. The method of claim 1, wherein said compound is administered over a time period of between about 1 and 30 minutes.

16. The method of claim 1, wherein said compound is administered in a sustained release formulation.

17. The method of claim 16, wherein said compound is administered over a time period of between about 1 and 30 days.

18. The method of claim 1, wherein the administration of the compound is accomplished by a method selected from the group consisting of injection, intramuscular injection, intravenous injection, implantation infusion device, inhalation, and transdermal diffusion.

19. The method of claim 12, wherein the compound is administered in a dosage of about 0.2 mg to about 100 mg.

20. The method of claim 1, wherein said compound is administered orally.

21. The method of claim 20, wherein said compound is in a form selected from the group consisting of capsules, tablets, granules, sprays, and syrups.

22. The method of claim 1, further comprising the step of administering an additional compound selected from the group consisting of an anti-inflammatory drug, a pain reliever, and a muscle relaxant.

23. A method for treating muscle pain associated with reactive oxygen species (ROS)-mediated oxidative damage, comprising:
identifying a subject with muscle pain associated with ROS-meditated oxidative damage; and
administering a compound effective to reduce the amount of ROS in said individual.

24. A method for inhibiting reactive oxygen species (ROS)-mediated oxidative damage to skeletal muscles of a subject comprising:
identifying an individual suffering from a disorder affecting muscle function that is caused or exacerbated by ROS-mediated oxidative damage; and
administering in said individual a compound effective to reduce the amount of ROS.
